(19) United States
(12) Patent Application Publication
(10) Pub. No.: US 2024/0420823 A1
(43) Pub. Date: Dec. 19, 2024

(54) MULTI-TYPE DATA MODIFICATION TECHNIQUES FOR DIETARY RESTRICTION DATA

(71) Applicant: Healthvice Inc., Woodstock, GA (US)

(72) Inventors: Ari Pekka Noso, Espoo (FI); Jaclynn Beth Morgan, Woodstock, GA (US); Albert Manninen, Espoo (FI)

(73) Assignee: Healthvice Inc., Woodstock, GA (US)

(21) Appl. No.: 18/745,416

(22) Filed: Jun. 17, 2024

Related U.S. Application Data
(60) Provisional application No. 63/508,441, filed on Jun. 15, 2023.

(51) Int. Cl. G16H 20/60; G06Q 50/12

(52) U.S. Cl. CPC G16H 20/60; G06Q 50/12

(57) ABSTRACT

In a dietary modification computing system, a taste-restrictions combination neural network generates a taste-restrictions vector based on a combination of restriction profile data and taste profile data. Also, a recipe-restrictions combination neural network generates a recipe-restrictions vector based on a combination of the restriction profile data and recipe data. An entity-recipe relational recommendation module generates entity-recipe prediction data that describing similarity among the taste-restrictions vector and the recipe-restrictions vector. An entity-recipe relational modification module generates optimization data that identifies an impact of the recipe data on the similarity. Based on the impact data identified by the optimization data, the dietary modification computing system generates modified recipe data that includes substitution recipe data and omits additional recipe data associated with the impact data. The dietary modification computing system provides the modified recipe data to one or more additional computing systems.

### RELATED APPLICATIONS

The present application claims priority to U.S. provisional application No. 63/508,441 for “Systems and methods for creating personalized recipes, shopping lists, and food calendars to accommodate medical and dietary restrictions” filed Jun. 15, 2023, which is incorporated by reference herein in its entirety.

### TECHNICAL FIELD

This disclosure relates generally to the field of neural network applications, and more specifically relates to combinations of artificial intelligence techniques to modify data.

### BACKGROUND

Dietary restrictions have multiple impacts on areas of health and resource consumption. In some cases, a person or group of people affected by dietary restrictions—such as food allergies, chronic disease, religious requirements, or other restriction types—may struggle to acquire sufficient nutrition that does not violate any of the dietary restrictions. For a person affected by dietary restrictions, ingesting a restricted ingredient could cause discomfort, illness, or potentially death. The example person may wish to find additional ingredients that provide adequate nutrition while still fulfilling the dietary restriction. In addition, dietary restrictions can affect resource consumption, such as food waste, transportation costs, household economic choices, or other impacts associated with resource consumption. In some cases, contemporary tools for identifying data (e.g., ingredient data) that violates dietary restrictions may fail to identify a particular ingredient that is identified by an alternative name (e.g., “sesame seeds” as compared to “tahini”). In addition, contemporary tools for identifying data that violates dietary restrictions may fail to provide an adequate alternative that fulfills a dietary restriction for a person while also being acceptable for the person's taste preferences.

It is desirable to provide computer-implemented tools that can accurately identify data describing ingredients that could violate a dietary restriction for a particular person or group of people. In addition, it is desirable to provide computer-implemented tools configured to generate modified data describing substitution ingredients that fulfill a dietary restriction.

### SUMMARY

According to certain embodiments, a dietary modification computing system includes a taste-restrictions combination neural network and a recipe-restrictions combination neural network. The taste-restrictions combination neural network generates an entity taste-restrictions vector based on a combination of entity restriction profile data and entity taste profile data. The recipe-restrictions combination neural network generates an entity recipe-restrictions vector based on a combination of entity restriction profile data and recipe data. An entity-recipe relational recommendation module in the dietary modification computing system generates entity-recipe prediction data that describes a calculated degree of multi-dimensional similarity among the entity taste-restrictions vector and the entity recipe-restrictions vector. An entity-recipe relational modification module in the dietary modification computing system generates optimization data that identifies an impact of a portion of the recipe data on the multi-dimensional similarity among the entity taste-restrictions vector and the entity recipe-restrictions vector. Based on the optimization data, the dietary modification computing system generates modified recipe data that includes substitution recipe data and omits the portion of the recipe data identified by the optimization data. The dietary modification computing system provides the modified recipe data to one or more additional computing systems.

These illustrative embodiments are mentioned not to limit or define the disclosure, but to provide examples to aid understanding thereof. Additional embodiments are discussed in the Detailed Description, and further description is provided there.

### BRIEF DESCRIPTION OF THE DRAWINGS

Features, embodiments, and advantages of the present disclosure are better understood when the following Detailed Description is read with reference to the accompanying drawings, where:

FIG. 1 is a block diagram depicting an example of a computing environment in which a dietary modification computing system generates entity-specific modified recipe data based on a combination of multiple data inputs, according to certain embodiments;

FIG. 2 is a flow chart depicting an example process for generating modified recipe data with multiple types of modified data, according to certain embodiments;

FIG. 3 is a diagram depicting an example data flow for multi-stage training processes for one or more neural networks included in a dietary modification computing system, according to certain embodiments; and

FIG. 4 is a block diagram depicting an example of a computing system for implementing a dietary modification computing system, according to certain embodiments.

### DETAILED DESCRIPTION

As discussed above, contemporary computer-based nutrition-planning tools configured for identifying ingredients that could violate dietary restrictions do not provide data describing potential substitution ingredients that fulfill a dietary restriction for a person or group of people while also being acceptable for the taste preferences of the person or group of people. Thus, contemporary computer-based nutrition-planning tools may fail to accurately predict recipes (or modifications to recipes) that fulfill both taste preferences and also dietary restrictions for the person or group of people. In addition, contemporary computer-based nutrition-planning tools do not generate modified recipe data that describes modified preparation techniques for a modified ingredient. Thus, contemporary computer-based nutrition-planning tools may fail to provide adequate information to both substitute an ingredient and also to prepare the substitution ingredient.

Certain embodiments described herein provide for a dietary modification computing system that is configured to generate modified recipe data, such as recipe data that is modified to include an ingredient or a preparation technique that fulfill a taste preference, dietary restriction, and adequate nutrition intake for an entity (e.g., a person or group of people). In some cases, the modified recipe data includes one or more of modified ingredient data, modified ingredient quantity data, modified preparation technique data, or modified presentation data. In addition, the dietary modification computing system is configured to provide the modified recipe data to one or more additional computing systems, such as a user device or an automated ordering computing system.

The following examples are provided to introduce certain embodiments of the present disclosure. A dietary modification computing system includes a taste-restrictions combination neural network and a recipe-restrictions combination neural network. Each of the taste-restrictions combination neural network and the recipe-restrictions combination neural network are configured to generate respective vector representations for various combinations of dietary restriction data, taste preference data, and recipe data. For example, the taste-restrictions combination neural network generates an entity taste-restrictions vector representing a combination of dietary restriction data and taste preference data for a particular entity. In addition, the recipe-restrictions combination neural network generates an entity recipe-restrictions vector representing a combination of the dietary restriction data for the particular entity and recipe data, such as recipe data describing a particular meal item. The dietary modification computing system includes an entity-recipe relational recommendation module that is configured to generate entity-recipe prediction data based on a combination of the entity taste-restrictions vector and the entity recipe-restrictions vector. In addition, the dietary modification computing system includes an entity-recipe relational modification module that is configured to generate optimization data describing modifications to the recipe data. The optimization data describes, for example, a substitution ingredient that replaces a particular ingredient omitted from the recipe data. The dietary modification computing system generates modified recipe data based on the optimization data. For example, the dietary modification computing system modifies ingredient data to describe the substitution ingredient and to omit the particular ingredient. In addition, the dietary modification computing system modifies preparation technique data to describe a substitution preparation technique for the substitution ingredient and to omit a particular preparation technique for the particular ingredient. In some cases, the dietary modification computing system modifies ingredient quantity data to describe a substitution measurement for the substitution ingredient. In some cases, the dietary modification computing system modifies presentation data to describe a substitution presentation for the substitution ingredient, such as an image or video that depicts the substitution ingredient being prepared according to the substitution preparation technique.

In this example, the entity-recipe relational recommendation module and the entity-recipe relational modification module generate respective data based on respective high-dimension analysis techniques. For example, the entity-recipe relational recommendation module generates the entity-recipe prediction data by calculating multi-dimensional similarities (or dissimilarities) among multiple high-dimension (e.g., 100 or more dimensions) data objects included in the entity taste-restrictions vector and the entity recipe-restrictions vector. In addition, the entity-recipe relational modification module generates the optimization data by applying high-dimension Jacobian matrix analysis to calculate an impact of each particular data value in the multiple high-dimension data objects included in the entity taste-restrictions vector and the entity recipe-restrictions vector.

As used herein, “high-dimension,” “high-dimensional,” and the like refer to computer-implemented data structures having a quantity of dimensions that is sufficiently large as to exceed human calculation, for example, a matrix data structure having 100 or more dimensions.

As used herein, the term “dietary restriction” refers to a requirement or recommendation to increase or reduce consumption of one or more food items (e.g., ingredients) for a person or group of people. Examples of dietary restrictions can include restrictions associated with health outcomes (e.g., improved nutrition, allergens, medication interactions with food), religious requirements (e.g., kosher, halal), lifestyle guidelines (e.g., vegetarian diet, high-performance athletic nutrition), or other characteristics associated with dietary restrictions. In some cases, a particular dietary restriction includes multiple ingredients (or other recipe components), such as a dietary restriction to increase consumption of spinach in combination with citrus (e.g., a dietary restriction related to increased iron consumption). In some cases, a particular dietary restriction includes a preparation technique of one or more ingredients, such as a dietary restriction to avoid raw or undercooked food items (e.g., a dietary restriction related to reducing complications during pregnancy). A dietary restriction can be negative, such as an ingredient, preparation technique, or other recipe component that should be omitted or reduced in consumption by a particular person or group of people. Examples of negative dietary restrictions can include restrictions associated with allergens (e.g., peanuts, gluten, lactose) or other types of ingredients (or other recipe components) that could cause a negative outcome for a particular person or group of people. A dietary restriction can be positive, such as an ingredient, preparation technique, or other recipe component that should be included or increased in consumption by a particular person or group of people. Examples of positive dietary restrictions can include restrictions associated with balanced nutritional intake, micronutrients (e.g., iron, folic acid), macronutrients (e.g., protein, fiber), or other types of ingredients (or other recipe components) that could cause a positive outcome for a particular person or group of people.

As used herein, the term “taste preference” refers to a preference by a person or group of people to include or avoid consumption of one or more food items (e.g., ingredients). Examples of taste preferences can include preferences associated with flavor, texture, aroma, effect (e.g., an intoxication effect from alcohol), or other characteristics associated with taste preferences. In some cases, a particular taste preference includes multiple ingredients (or other recipe components), such as a taste preference for salt combined with chocolate. In some cases, a particular taste preference includes a preparation technique of one or more ingredients, such as a taste preference for fried food items. A taste preference can be negative, such as an ingredient, preparation technique, or other recipe component that a particular person or group of people prefers to avoid or reduce in consumption. Examples of negative taste preferences can include preferences associated with unpalatable ingredients (e.g., bitter flavors), a particular preparation technique (e.g., fermentation), or other types of ingredients (or other recipe components) that are disliked by a particular person or group of people. A taste preference can be positive, such as an ingredient, preparation technique, or other recipe component that a particular person or group of people prefers to increase in consumption. Examples of positive taste preferences can include preferences associated with unpalatable ingredients (e.g., sweet flavors), a particular preparation technique (e.g., roasting), or other types of ingredients (or other recipe components) that are enjoyed by a particular person or group of people.

Certain embodiments described herein provide improved techniques for accurately identifying substitution ingredients that fulfill both taste preferences and dietary restrictions for a person or group of people. In addition, certain embodiments described herein provide improved techniques for generating new recipe data that accurately modifies substitution ingredients and also substitution preparation techniques. For example, a dietary modification computing system can utilize particular rules to combine multiple high-dimension vector data objects that are generated by multiple neural networks. In addition, the dietary modification computing system can utilize particular rules to generate new data objects, such as modified ingredient data or modified preparation technique data. In some cases, a dietary modification computing system that utilizes the techniques described herein can provide an entity with more accurate information to prepare a modified recipe that successfully fulfills both taste preferences and dietary restrictions for the entity. In some cases, the dietary modification computing system utilizing the techniques described herein can generate accurate data to help the entity avoid or reduce consumption of ingredients that violate dietary restrictions for the entity. In some cases, the dietary modification computing system utilizing the techniques described herein can identify substitution ingredients and substitution preparation techniques which can resolve conflict among multiple dietary restrictions for the entity. For example, if a person (or group of people) has received a first dietary restriction (such as a medical recommendation from a medical doctor) to decrease red meat consumption and a second dietary restriction (such as an athletic recommendation from a sport nutritionist) to increase protein consumption, the person may experience conflict between the first and second dietary restrictions. In this example, the dietary modification computing system utilizing the techniques described herein can identify substitution ingredients and substitution preparation techniques to resolve the conflict among the first and second dietary restrictions, such as identifying a substitution ingredient fish and a substitution preparation technique of baking. Continuing with this example, the dietary modification computing system utilizing the techniques described herein can generate modified recipe data that describes the substitution ingredient and the substitution preparation technique, allowing the person to benefit from the identified substitutions.

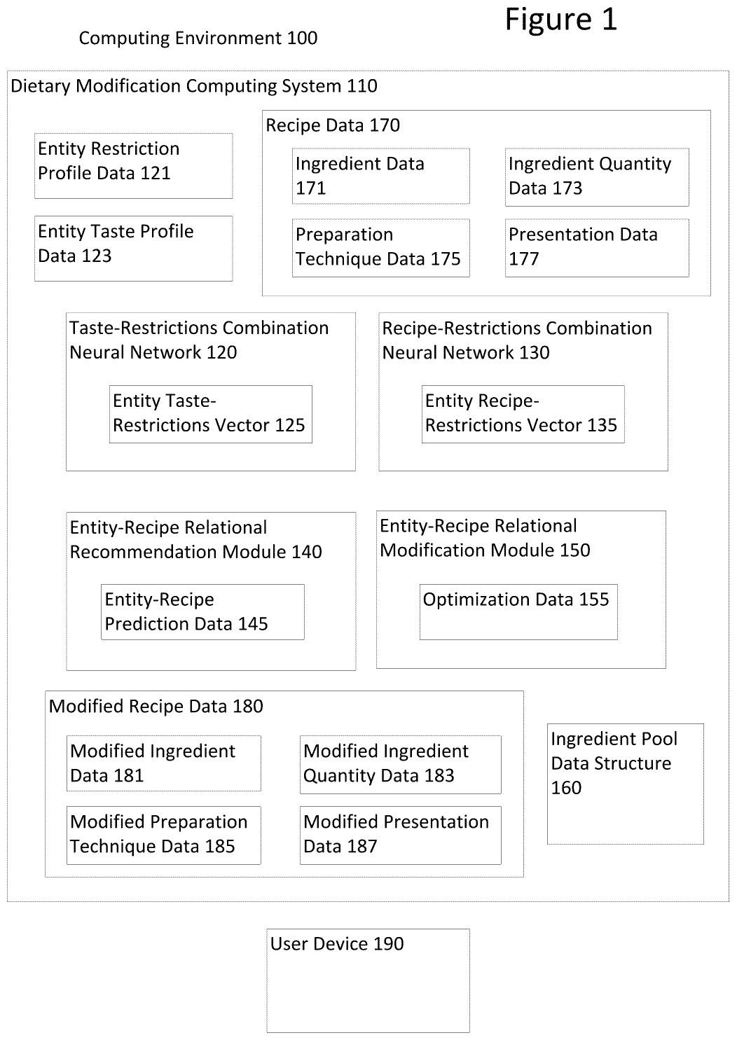

Referring now to the drawings, FIG. 1 is a diagram depicting an example of a computing environment 100, in which a dietary modification computing system 110 generates entity-specific modified recipe data based on a combination of multiple data inputs. In the computing environment 100, the multiple data inputs are generated via respective artificial intelligence techniques that are implemented by respective components of the dietary modification computing system 110. In addition, the dietary modification computing system 110 provides the entity-specific modified recipe data to one or more additional computing devices, such as a user device 190 included in the computing environment 100. In some cases, the dietary modification computing system 110, the user device 190, and one or more additional computing systems are configured to exchange data via one or more computing networks, such as a local or global area network. In FIG. 1, the user device 190 is associated with a particular entity, such as a person who uses the user device 190, a family group of people (e.g., a household) who interact with the user device 190, or other groups of people.

In the computing environment 100, the dietary modification computing system 110 includes one or more of a taste-restrictions combination neural network 120, a recipe-restrictions combination neural network 130, an entity-recipe relational recommendation module 140, or an entity-recipe relational modification module 150 (also referred to herein as “relational modification module 150”). In the dietary modification computing system 110, each of the taste-restrictions combination neural network 120 and the recipe-restrictions combination neural network 130 are configured to apply one or more artificial intelligence techniques to generate vector representations of particular combinations of data types. In addition, the entity-recipe relational recommendation module 140 is configured to identify a relationship between (or among) multiple data objects that are generated as outputs from the taste-restrictions combination neural network 120 and the recipe-restrictions combination neural network 130. Furthermore, the entity-recipe relational modification module 150 is configured to modify the relationship identified by the entity-recipe relational recommendation module 140, such as by modifying one or more data objects based on probability data that is generated as an output from the entity-recipe relational recommendation module 140. FIG. 1 depicts the taste-restrictions combination neural network 120, the recipe-restrictions combination neural network 130, the entity-recipe relational recommendation module 140, and the entity-recipe relational modification module 150 as being included in the dietary modification computing system 110, but other implementations are possible. For example, one or more additional computing systems configured to generate vector representations of data type combinations could provide one or more vector data objects to the dietary modification computing system 110 via a communicative connection.

In FIG. 1, the dietary modification computing system 110 receives (or can otherwise access) one or more of entity restriction profile data 121 or entity taste profile data 123. The entity restriction profile data 121 includes data values that indicate one or more dietary restrictions for the entity that is associated with the user device 190. In addition, the entity taste profile data 123 includes data values that indicate one or more taste preferences for the entity that is associated with the user device 190. In some cases, the dietary modification computing system 110 receives at least a portion of the entity restriction profile data 121 or the entity taste profile data 123 from the user device 190. For example, a user could provide, via a user interface component of the user device 190, a portion of the entity restriction profile data 121 describing one or more dietary restrictions of the associated entity, such as food allergies (or additional allergies), an interaction between a prescription medication and an ingredient, a medical recommendation to increase a particular nutrient consumption, or other types of dietary restrictions. In addition, the user could provide, via the user interface component of the user device 190, a portion of the entity taste profile data 123 describing one or more taste preferences of the associated entity, such as an enjoyment of fresh fruit, a dislike of spicy foods, or other types of taste preferences. In some cases, the entity restriction profile data 121 or the entity taste profile data 123 are associated with an entity that includes a particular person, such as a person who uses the user device. In some cases, the entity restriction profile data 121 or the entity taste profile data 123 are associated with an entity that includes multiple people, such as a family, a household group (e.g., roommates), a student group (e.g., classmates), or other types of multi-person groups.

In some implementations, the entity restriction profile data 121 includes data describing one or more dietary restrictions for the entity associated with the user device 190, such as positive dietary restrictions, negative dietary restrictions, or a combination of positive and negative dietary restrictions. In some cases, the entity restriction profile data 121 includes data describing a threshold value (or values) associated with a dietary restriction. As an example, the entity restriction profile data 121 could include first text data describing a negative dietary restriction prohibiting peanut consumption and first numeric data describing a threshold value of 0 (e.g., any amount of peanuts is prohibited for the example entity). As another example, the entity restriction profile data 121 could include second text data describing a positive dietary restriction to increase dietary fiber consumption and second numeric data describing a threshold value range of 15-20 (e.g., daily consumption of 15-20 grams of dietary fiber is encouraged for the example entity). In addition, the entity taste profile data 123 includes data describing one or more taste preferences for the entity associated with the user device 190, such as positive taste preferences, negative taste preferences, or a combination of positive and negative taste preferences. In some cases, the entity taste profile data 123 includes data describing a threshold value (or values) associated with a taste preference. As another example, the entity taste profile data 123 could include third text data describing a positive taste preference for chili peppers and third numeric data (or other data types) describing a threshold value range (e.g., the example entity enjoys a small amount of spicy flavor, but not a large amount).

In FIG. 1, the dietary modification computing system 110 receives (or can otherwise access) recipe data 170, such as data describing a recipe for a particular meal item. The recipe data 170 includes data values that indicate one or more portions of recipe data, such as one or more of ingredient data 171, ingredient quantity data 173 (also referred to herein as “quantity data 173”), preparation technique data 175, or presentation data 177. In FIG. 1, the ingredient data 171 includes data, such as text data, describing each ingredient that is included in the meal item described by the recipe data 170. In addition, the quantity data 173 includes data, such as numeric data, describing a respective quantity of each ingredient described by the ingredient data 171. Furthermore, the preparation technique data 175 includes data, such as text data, describing one or more techniques to prepare (e.g., marinate, chop, boil, combine) each ingredient described by the ingredient data 171. In some cases, the presentation data 177 includes data indicating one or more presentation characteristics of the recipe data 170. For example, the presentation data 177 can indicate a language characteristic that indicates a language for presenting the ingredient data 171, a measurement characteristic indicating a measurement system (e.g., metric, imperial) for the quantity data 173, a visual preparation characteristic indicating that the preparation technique data 175 includes or is modified to include visual depictions (e.g., images, video) of preparation techniques described by the data 175, or other suitable presentation characteristics for recipe data. In addition, the presentation data 177 can include one or more data types, such as image data, video data, audio data, text data, numeric data, or any other data type (or combination of data types) suitable for presenting recipe data. In some cases, the dietary modification computing system 110 receives at least some of the recipe data 170 from an additional computing system, such as the user device 190 or another computing system including a database of recipe data. FIG. 1 describes the recipe data 170 as including data describing a particular recipe (e.g., a particular meal item), but other implementations are possible. For example, the dietary modification computing system 110 could receive recipe data that describes multiple recipes, such as for a meal (e.g., entree, side dish), a meal plan (e.g., weekly set of meals, monthly set of meals), or other sets of multiple recipes.

In the computing environment 100, the taste-restrictions combination neural network 120 generates an entity taste-restrictions vector 125 based on a combination of data. For example, the taste-restrictions combination neural network 120 receives one or more of the entity restriction profile data 121 or the entity taste profile data 123 as input data objects. Based on the input data objects, the taste-restrictions combination neural network 120 generates the entity taste-restrictions vector 125, such as by applying one or more artificial intelligence techniques to generate a vector representation of the combination of the entity restriction profile data 121 with the entity taste profile data 123. In some cases, the entity taste-restrictions vector 125 includes numeric data (or other types of data) describing relationships (or other combinations) among data values from the input data objects. For example, the entity taste-restrictions vector 125 can be a data structure arranged as a high-dimensional (e.g., 100 or more dimensions) matrix in which data values of the matrix entries identify relationships among data values from the entity restriction profile data 121 and the entity taste profile data 123. In some cases, the taste-restrictions combination neural network 120 determines a relationship among first data values from the entity restriction profile data 121 describing one or more dietary restrictions and second data values from the entity taste profile data 123 describing one or more taste preferences. As an example, the taste-restrictions combination neural network 120 can determine a first relationship among dietary restriction data values indicating a low-threshold dietary restriction on nuts (e.g., an intense nut allergy) and taste preference data values indicating a low preference for nuts. In this example, the first relationship determined by the taste-restrictions combination neural network 120 is a taste-restrictions relationship correlated strongly with omitting nuts. As an additional example, the taste-restrictions combination neural network 120 can determine a second relationship among dietary restriction data values indicating a mid-threshold dietary restriction on tomatoes (e.g., a mild tomato allergy) and taste preference data values indicating a high preference for tomatoes. In this additional example, the second relationship determined by the taste-restrictions combination neural network 120 is an additional taste-restrictions relationship correlated mildly with omitting tomatoes, e.g., a person has a mild tomato allergy but enjoys the flavor enough to sometimes consume meals with tomatoes. In some cases, the entity taste-restrictions vector 125 can include multiple dimensions for a particular taste-restrictions data combination, such as dimensions including data values for a taste-restrictions data combination with multiple variations (e.g., multiple species of an ingredient, multiple types of preparations, etc.).

In the computing environment 100, the recipe-restrictions combination neural network 130 generates an entity recipe-restrictions vector 135 based on an additional combination of data. For example, the recipe-restrictions combination neural network 130 receives the entity restriction profile data 121 and the recipe data 170 as input data objects. Based on the input data objects, the recipe-restrictions combination neural network 130 generates the entity recipe-restrictions vector 135, such as by applying one or more artificial intelligence techniques to generate a vector representation of the combination of the entity restriction profile data 121 with the recipe data 170. In some cases, the entity recipe-restrictions vector 135 includes numeric data (or other types of data) describing relationships (or other combinations) among data values from the input data objects. For example, the entity recipe-restrictions vector 135 can be a data structure arranged as a high-dimensional (e.g., 100 or more dimensions) matrix in which data values of the matrix entries identify relationships among data values from the entity restriction profile data 121 and the recipe data 170. In FIG. 1, the entity taste-restrictions vector 125 and the entity recipe-restrictions vector 135 have a same number of dimensions, e.g., both are n-dimensional, but other implementations are possible. In some cases, the recipe-restrictions combination neural network 130 determines a relationship among third data values from the entity restriction profile data 121 describing one or more dietary restrictions and fourth data values from the recipe data 170 describing one or more ingredients, ingredient quantities, or preparation techniques. As an example, the recipe-restrictions combination neural network 130 can determine a third relationship among dietary restriction data values indicating a medical recommendation to increase consumption of fresh fruit and recipe data values indicating a relatively high proportion of ingredients identified as fruit. In this example, the third relationship determined by the recipe-restrictions combination neural network 130 is a recipe-restrictions relationship correlated strongly with a nutrition benefit for the example person (or other entity).

FIG. 1 describes generating the entity taste-restrictions vector 125 based on a combination of the entity restriction profile data 121 with the entity taste profile data 123, and generating the entity recipe-restrictions vector 135 based on a combination of the entity restriction profile data 121 with the recipe data 170, but other implementations are possible. For example, a dietary modification computing system could generate (or receive) one or more vector representations of taste preference data, dietary restriction data, or recipe data that are uncombined.

In the dietary modification computing system 110, the entity-recipe relational recommendation module 140 receives one or more of the entity taste-restrictions vector 125 or the entity recipe-restrictions vector 135 as input vector data objects. Based on one or more relationships identified among the input vector data objects, the entity-recipe relational recommendation module 140 generates entity-recipe prediction data 145. In some cases, the entity-recipe relational recommendation module 140 generates the entity-recipe prediction data 145 by applying one or more comparative analysis techniques, such as principal component analysis (“PCA”), to calculate a similarity between (or among) the entity taste-restrictions vector 125 and the entity recipe-restrictions vector 135. For example, based on multiple high-dimensional (e.g., 100 or more dimensions) matrix data objects included in the entity taste-restrictions vector 125 and the entity recipe-restrictions vector 135, the entity-recipe relational recommendation module 140 calculates multi-dimensional similarities (or dissimilarities) among the vectors 125 and 135. In addition, the entity-recipe relational recommendation module 140 calculates probability data indicating a relative effectiveness of the recipe data 170, such as a relative effectiveness in addressing the combination of taste preferences and dietary restrictions described by the entity restriction profile data 121 and the entity taste profile data 123. In some cases, a relatively high degree of multi-dimensional similarity among the entity taste-restrictions vector 125 and the entity recipe-restrictions vector 135 indicates a relatively high degree of overlap among data values describing taste-restrictions relationships and additional data values describing recipe-restrictions relationships, e.g., the taste-restrictions relationships and recipe-restrictions relationships are similar. In some cases, a relatively low degree of multi-dimensional similarity among the entity taste-restrictions vector 125 and the entity recipe-restrictions vector 135 indicates a relatively low degree of overlap among the data values describing taste-restrictions relationships and the additional data values describing recipe-restrictions relationships, e.g., the taste-restrictions relationships and recipe-restrictions relationships are dissimilar. Responsive to determining a degree of multi-dimensional similarity, the entity-recipe relational recommendation module 140 generates the entity-recipe prediction data 145. For example, the entity-recipe relational recommendation module 140 generates first probability data indicating a relatively high effectiveness of the recipe data 170 in response to determining a relatively high degree of multi-dimensional similarity, or second probability data indicating a relatively low effectiveness of the recipe data 170 in response to determining a relatively low degree of multi-dimensional similarity. In addition, the entity-recipe relational recommendation module 140 generates or modifies the entity-recipe prediction data 145 to include the first probability data or the second probability data. FIG. 1 describes the entity-recipe relational recommendation module 140 as generating the entity-recipe prediction data 145 based on a combination of the entity taste-restrictions vector 125 and the entity recipe-restrictions vector 135, but other implementations are possible. For example, an entity-recipe relational recommendation module (or other component of a dietary modification computing system) could generate prediction data based on one or more vector representations of taste preference data, dietary restriction data, or recipe data that are uncombined, such as by determining a degree of multi-dimensional similarity among recipe data, entity restriction profile data, and entity taste profile data.

In the dietary modification computing system 110, the entity-recipe relational modification module 150 receives one or more of the entity-recipe prediction data 145, the entity taste-restrictions vector 125, the entity recipe-restrictions vector 135, or the recipe data 170 as input data objects. Based on the input data objects, the entity-recipe relational modification module 150 generates optimization data 155 that describes one or more modifications to the recipe data 170. In some cases, the optimization data 155 describes at least one modification that increases an effectiveness of the recipe data 170, such as a relative effectiveness in addressing the combination of taste preferences and dietary restrictions described by the entity restriction profile data 121 and the entity taste profile data 123. In some cases, the entity-recipe relational modification module 150 generates the optimization data 155 responsive to determining that probability data included in the entity-recipe prediction data 145 fulfills (e.g., has a particular relationship with) an optimization threshold value. For example, the entity-recipe relational modification module 150 could determine that the entity-recipe prediction data 145 includes probability data having a value below the optimization threshold value, such as the above example of second probability data indicating a relatively low effectiveness of the recipe data 170. In response to determining that the probability data has a value below the optimization threshold value, the entity-recipe relational modification module 150 generates the optimization data 155 which describes at least one modification that increases an effectiveness of the recipe data 170. In an additional example, the entity-recipe relational modification module 150 could determine that the entity-recipe prediction data 145 includes probability data having a value that equals or exceeds the optimization threshold value, such as the above example of first probability data indicating a relatively high effectiveness of the recipe data 170. In this additional example, responsive to determining that the probability data has a value that equals or exceeds the optimization threshold value, the entity-recipe relational modification module 150 may omit generation of optimization data, or may generate or modify additional optimization data indicating few or no modifications to the recipe data 170.

In the computing environment 100, the entity-recipe relational modification module 150 generates the optimization data 155 describing one or more modifications to the recipe data 170, such as based on a determination probability data in the entity-recipe prediction data 145 is below (or otherwise fulfills) the optimization threshold value. In some cases, the entity-recipe relational modification module 150 generates the optimization data 155 by applying one or more vector-modification techniques, such as a high-dimensional Jacobian matrix analysis technique. For example, the entity-recipe relational modification module 150 determines that each of the entity taste-restrictions vector 125 and the entity recipe-restrictions vector 135 are high-dimensional and have a same number of dimensions, e.g., both are n-dimensional. In addition, the entity-recipe relational modification module 150 calculates a Jacobian determinant that is based on a combination of the entity taste-restrictions vector 125 and the entity recipe-restrictions vector 135. In addition, the entity-recipe relational modification module 150 identifies new data about the recipe data 170 by applying the high-dimensional Jacobian matrix analysis technique. For example, the entity-recipe relational modification module 150 generates impact data describing an impact of a particular data value in the entity taste-restrictions vector 125 on a correlated (e.g., correlated matrix position) data value in the entity recipe-restrictions vector 135. In some cases, the entity-recipe relational modification module 150 generates respective impact data for each particular pair (or other set) of correlated data values in the vectors 125 and 135. In addition, the impact data indicates an impact of each particular data value on the correlated particular data value. For example, based on calculated impact data between a particular correlated pair of a taste-restrictions data value and a recipe-restrictions data value, the entity-recipe relational modification module 150 identifies an impact of the taste-restrictions data value on the recipe-restrictions data value and an additional impact of the recipe-restrictions data value on the taste-restrictions data value. FIG. 1 describes the entity-recipe relational modification module 150 as generating the optimization data 155 via a high-dimensional Jacobian matrix analysis technique, but other implementations are possible, such as partial or total differentiation of one or more vector values, partial or total differentiation of one or more vector values identified as having a relatively high significance to an effectiveness of particular entity-recipe prediction data, or other suitable vector-modification techniques or combinations of techniques.

Based on the calculated Jacobian determinant, the entity-recipe relational modification module 150 identifies one or more portions of the recipe data 170 that can modify one or more relationships among the entity taste-restrictions vector 125 and the entity recipe-restrictions vector 135, such as a modification that increases a degree of multi-dimensional similarity among the vectors 125 and 135. For example, the entity-recipe relational modification module 150 identifies, based on the calculated Jacobian determinant that some or all of the ingredient data 171 is associated with low similarity among the entity taste-restrictions vector 125 and the entity recipe-restrictions vector 135. In some cases, the entity-recipe relational modification module 150 determines that the ingredient data 171 is associated with a data structure describing multiple ingredients, such as an ingredient pool data structure 160. In addition, the ingredient pool data structure 160 includes one or more ingredient data sets that include (or are otherwise associated with) the particular portion of the ingredient data 171 associated with low similarity of the vectors 125 and 135. As an example, the entity-recipe relational modification module 150 could identify that a particular portion of the ingredient data 171 describing peanuts is correlated with a low similarity of the vectors 125 and 135, e.g., the peanut ingredient data conflicts with dietary restriction data describing a peanut allergy. In this example, the entity-recipe relational modification module 150 determines that the ingredient pool data structure 160 includes one or more ingredient data sets associated with peanuts, such as a first ingredient data structure describing commonly associated allergens (e.g., peanuts, walnuts, almonds) and a second ingredient data structure describing potential allergen substitutions (e.g., sunflower seeds, pumpkin seeds).

In FIG. 1, the entity-recipe relational modification module 150 determines an additional portion of ingredient data from the ingredient pool data structure 160 that increases similarity among the entity taste-restrictions vector 125 and the entity recipe-restrictions vector 135. In addition, the entity-recipe relational modification module 150 calculates one or more optimizations of the calculated Jacobs matrix, such as an optimization that substitutes an ingredient data with additional ingredient data. In some cases, the entity-recipe relational modification module 150 determines an additional impact of the additional ingredient data on the effectiveness of the recipe data 170 by applying the one or more vector-modification techniques to the additional ingredient data, such as by calculating one or more additional Jacobian determinants for additional entity taste-restrictions vectors and entity recipe-restrictions vectors that include the additional ingredient data (and/or omit the ingredient data that conflicts with the dietary restriction data). In addition, the entity-recipe relational modification module 150 generates or modifies the optimization data 155 to include the additional ingredient data. Continuing with the above example, the entity-recipe relational modification module 150 determines that additional ingredient data describing sunflower seeds increases a degree of similarity among modified versions of the entity taste-restrictions vector 125 and the entity recipe-restrictions vector 135, e.g., a modification of the vector 135 to replace peanut ingredient data with sunflower seed ingredient data. In this example, the entity-recipe relational modification module 150 generates or modifies the optimization data 155 to include the sunflower seed ingredient data and omit peanut ingredient data. FIG. 1 describes modifying the optimization data 155 to include additional ingredient data and omit ingredient data that conflicts with dietary restriction data, but other types of modifications are possible, such as modifying ingredient quantity data, modifying preparation technique data, modifying ingredient data to include additional ingredient data without omitting other ingredients, or other types of modifications that can optimize recipe data for a particular entity.

In some cases, one or more of the taste-restrictions combination neural network 120 or the recipe-restrictions combination neural network 130 are prevented, e.g., by the dietary modification computing system 110, from accessing at least a portion of the entity restriction profile data 121, the entity taste profile data 123, or the recipe data 170. For example, the dietary modification computing system 110 can prevent the taste-restrictions combination neural network 120 from accessing the recipe data 170 (or nutrition data associated with the recipe data 170). In addition, the dietary modification computing system 110 can prevent the recipe-restrictions combination neural network 130 from accessing the entity taste profile data 123. In some cases, preventing one or more of the taste-restrictions combination neural network 120 or the recipe-restrictions combination neural network 130 from accessing a particular portion of data can improve accuracy of output data objects from the neural networks 120 or 130. For example, preventing the taste-restrictions combination neural network 120 from accessing the recipe data 170 (or associated nutrition data) can improve accuracy for taste information represented by an output of the taste-restrictions combination neural network 120, such as by keeping taste preferences separated from recipe data or nutrition data. In addition, preventing the recipe-restrictions combination neural network 130 from accessing the entity taste profile data 123 can improve accuracy for recipe or nutrition information represented by an output of the recipe-restrictions combination neural network 130, such as by keeping recipes and associated nutrition separated from taste preference data. In some cases, preventing one or more of the taste-restrictions combination neural network 120 or the recipe-restrictions combination neural network 130 from accessing a particular portion of data can improve accuracy of output data objects from additional components of the dietary modification computing system 110. For example, the entity-recipe relational modification module 150 can generate more accurate optimizations (e.g., improving the optimization data 155) based on inputs that omit confounding data, such as the entity taste-restrictions vector 125 that omits recipe data and nutrition data or the entity recipe-restrictions vector 135 that omits taste preference data.

Based on the optimization data 155 calculated by the entity-recipe relational modification module 150, the dietary modification computing system 110 generates or modifies the recipe data 170, such as by generating modified recipe data 180. In the dietary modification computing system 110, the modified recipe data 180 includes one or more of modified ingredient data 181, modified ingredient quantity data 183 (also referred to herein as “modified quantity data 183”), modified preparation technique data 185, or modified presentation data 187. In some cases, the dietary modification computing system 110 generates a portion of the modified recipe data 180 to include, omit, or otherwise modify a particular portion of ingredient data, quantity data, or preparation technique data described by the optimization data 155. Continuing with the above example, the dietary modification computing system 110 generates the modified ingredient data 181 to include the sunflower seed ingredient data and omit the peanut ingredient data. In addition, the dietary modification computing system 110 generates the modified quantity data 183 to include sunflower seed quantity data and omit peanut quantity data.

In some cases, the dietary modification computing system 110 generates a portion of the modified recipe data 180 based on additional data received from one or more additional computing systems. For example, the dietary modification computing system 110 identifies, in the optimization data 155, ingredient data describing one or more additional or omitted ingredients in the recipe data 170. Responsive to identifying the one or more additional or omitted ingredients, the dietary modification computing system 110 may provide request data to an additional computing system that is configured to provide a large language model (“LLM”). For example, the additional computing system could include an LLM configured to generate or otherwise provide additional preparation technique data for particular ingredient data, e.g., one or more substitution preparation techniques for one or more substitution ingredients. In addition, the dietary modification computing system 110 receives, from the additional computing system, the additional preparation technique data that is generated by the LLM. Based on the additional preparation technique data received from the additional computing system, the dietary modification computing system 110 generates the modified preparation technique data 185 to include the additional preparation technique data. In some cases, responsive to receiving the additional preparation technique data received from the additional computing system, the dietary modification computing system 110 generates (or modifies) the modified preparation technique data 185 to omit a portion of the preparation technique data 175 that is associated with an omitted ingredient. For example, responsive to determining that the optimization data 155 indicates omitting one or more portions of ingredient data, e.g., omitting peanut ingredient data, the dietary modification computing system 110 omits one or more associated portions of preparation technique data from the modified preparation technique data 185, e.g., omitting preparation technique data which describes chopping peanuts. In some implementations, the dietary modification computing system 110 receives multiple portions of additional data received from multiple additional computing systems. For example, the dietary modification computing system 110 may provide the request data (or additional request data) to a further computing system that is configured to provide an image generation model configured to generate or otherwise provide additional presentation data (e.g., images, video) for particular ingredient data, quantity data, or preparation technique data. In addition, the dietary modification computing system 110 receives, from the further computing system, the additional presentation data that is generated by the image generation model. Based on the additional presentation data received from the further computing system, the dietary modification computing system 110 generates the modified presentation data 187 to include the additional presentation data, to omit a portion of the presentation data 177 associated with an omitted ingredient, or other suitable adjustments to the modified presentations data 187.

In FIG. 1, the dietary modification computing system 110 provides the modified recipe data 180 to a computing system, such as the user device 190. The user device 190 is configured to present the modified recipe data 180 via a user interface, such as on a display device or by an audio device. In some cases, the user device 190 is configured to provide alert data corresponding to the modified recipe data 180, such as an alert that an ingredient, quantity, preparation technique, or other portion of the recipe data 170 is modified. In some implementations, the dietary modification computing system 110 receives one or more portions of feedback data from the user device 190. In addition, the dietary modification computing system 110 trains (or modifies training) of one or more of the taste-restrictions combination neural network 120 or the recipe-restrictions combination neural network 130 based on the received feedback data. For example, the dietary modification computing system 110 (or a component thereof) may calculate one or more loss functions based on the received feedback data, such as a loss function indicating a difference (or other relationship) between the received feedback data and one or more of the entity taste-restrictions vector 125 or the entity recipe-restrictions vector 135.

In some implementations, the dietary modification computing system 110 provides the modified recipe data 180 to one or more additional computing systems, such as an additional computing system in addition or alternatively to the user device 190. For example, the dietary modification computing system 110 can be configured to communicate (e.g., via one or more computing networks) with an additional computing system that is configured to generate grocery orders, such as an automated ordering computing system associated with a grocery store or a restaurant. In addition, the dietary modification computing system 110 can provide the modified recipe data 180 (or a portion thereof, such as the modified ingredient data 181 or the modified quantity data 183) to the automated ordering computing system. In some cases, the dietary modification computing system 110 can provide additional data to the automated ordering computing system, such as an order delivery date or a reservation date (e.g., at the associated restaurant). In response to receiving the modified recipe data 180 from the dietary modification computing system 110, the automated ordering computing system modifies order data to include one or more portions of the modified recipe data 180. For example, the automated ordering computing system modifies the order data to include a substitution ingredient from the modified ingredient data 181 and to omit a particular ingredient from the recipe data 170 (e.g., the particular ingredient being substituted). In some cases, the automated ordering computing system requests additional data from the dietary modification computing system 110 in response to receiving the modified recipe data 180, such as requesting one or more options for substitution ingredients. In addition, the dietary modification computing system 110 can respond to a request from the automated ordering computing system by identifying, from the ingredient pool data structure 160, one or more substitution ingredients associated with the modified recipe data 180, and providing data describing the identified substitution ingredients to the automated ordering computing system. In some cases, the automated ordering computing system is configured to modify additional data, such as delivery schedule data, in response to receiving the modified recipe data 180. In another implementation, the dietary modification computing system 110 provides the modified recipe data 180 to an additional computing system configured to modify or generate a shopping list, such as a shopping list that incorporates data about ingredients already available to an entity. For example, the dietary modification computing system 110 can be configured to communicate with one or more computer-based appliances (e.g., “smart refrigerator,” IoT-enabled appliances) that are configured to identify ingredients present in (or near to) the appliance. In addition, in response to receiving the modified recipe data 180 from the dietary modification computing system 110, the one or more computer-based appliances can be configured to modify order data (such as a shopping list) to omit ingredients already present and include ingredients not identified by the computer-based appliances.

In some implementations, providing the modified recipe data 180 to the automated ordering computing system (or other additional computing system) can improve efficient operations of an organization associated with the automated ordering computing system. For instance, based on improved information about a combined effect of taste preferences and dietary restrictions, the example grocery store or restaurant can decrease expenditure for resources related to transportation and storage of ordered items, or increase efficient use of ordered items, such as by decreasing food waste related to disliked or restricted ingredients.

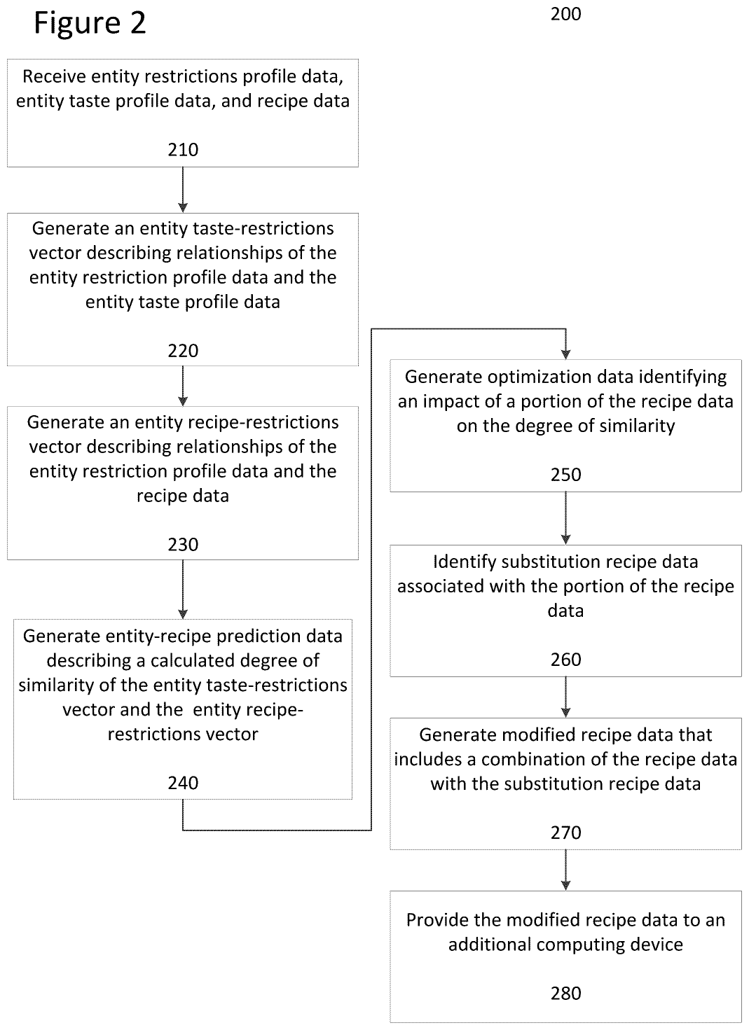

FIG. 2 is a flow chart depicting an example of a process 200 for generating modified recipe data with multiple types of modified data. In some embodiments, such as described in regards to FIG. 1, a computing device executing a dietary modification computing system implements operations described in FIG. 2, by executing suitable program code. For illustrative purposes, the process 200 is described with reference to the examples depicted in FIG. 1. Other implementations, however, are possible.

At block 210, the process 200 involves receiving, by a dietary modification computing system, one or more of entity restrictions profile data, entity taste profile data, or recipe data. In some cases, one or more of the entity restrictions profile data or the entity taste profile data is associated with an entity, such as a person or a group of people. In addition, the dietary modification computing system receives one or more of the data types from one or more additional computing systems. For example, the dietary modification computing system 110 receives one or more of the entity restrictions profile data 121, the entity taste profile data 123, or the recipe data 170 from the user device 190. In some cases, the entity restrictions profile data includes data values that indicate one or more dietary restrictions for the entity. In addition, the entity taste profile data includes data values that indicate one or more taste preferences for the entity.

At block 220, the process 200 involves generating, based on a combination of the entity restrictions profile data and the entity taste profile data, one or more high-dimension taste-restrictions data objects associated with the entity, such as an entity taste-restrictions vector. In some cases, a component of the dietary modification computing system generates the entity taste-restrictions vector, such as a taste-restrictions combination neural network or other type of module configured to combine multiple types of data. In some cases, the taste-restrictions combination neural network (or other type of module) generates the entity taste-restrictions vector as a high-dimension matrix data structure that includes data values identifying relationships of the entity restrictions profile data and the entity taste profile data. For example, the taste-restrictions combination neural network 120 generates the entity taste-restrictions vector 125 based on a combination of the entity restrictions profile data 121 and the entity taste profile data 123.

At block 230, the process 200 involves generating, based on a combination of the entity restrictions profile data and the recipe data, one or more high-dimension recipe-restrictions data objects associated with the entity, such as an entity recipe-restrictions vector. In some cases, a component of the dietary modification computing system generates the entity recipe-restrictions vector, such as a recipe-restrictions combination neural network or other type of module configured to combine multiple types of data. In some cases, the recipe-restrictions combination neural network (or other type of module) generates the entity recipe-restrictions vector as a high-dimension matrix data structure that includes data values identifying relationships of the entity restrictions profile data and the recipe data. For example, the recipe-restrictions combination neural network 130 generates the entity recipe-restrictions vector 135 based on a combination of the entity restrictions profile data 121 and the recipe data 170.

The process 200 is described as involving generating multiple vector data objects that are each based on a respective combination of data types, but other implementations are possible. For example, the example dietary modification computing system could include one or more modules configured to generate respective vector representations of the entity restrictions profile data, the entity taste profile data, and the recipe data. In addition, the example dietary modification computing system can generate multiple vector representations that, respectively, include combined or uncombined data types. As an example, the dietary modification computing system could include one or more modules configured to generate a first vector representing combined restrictions profile data and taste profile data, and a second vector representing uncombined recipe data. As another example, the dietary modification computing system could include one or more modules configured to generate a third vector representing uncombined restrictions profile data, a fourth vector representing uncombined taste profile data, and a fifth vector representing uncombined recipe data. Additional implementations for additional vectors, e.g., representing combined or uncombined data types, could be utilized.

At block 240, the process 200 involves generating entity-recipe prediction data, such as by an entity-recipe relationship recommendation module included in the dietary modification computing system. In some cases, the entity-recipe prediction data includes probability data indicating, for the recipe data, a relative effectiveness in addressing a combination of taste preferences and dietary restrictions for the entity, such as taste preferences and dietary restrictions described by the entity restriction profile data and the entity taste profile data. In some cases, the entity-recipe relationship recommendation module generates the entity-recipe prediction data by calculating multi-dimensional similarities (or dissimilarities) among the entity taste-restrictions vector and the entity recipe-restrictions vector. For example, the entity-recipe relational recommendation module 140 generates the entity-recipe prediction data 145 by calculating multi-dimensional similarities among the entity taste-restrictions vector 125 or the entity recipe-restrictions vector 135.

The process 200 is described as involving generating entity-recipe prediction data based on multiple vector data objects representing respective combinations of data types, but other implementations are possible. For example, the example dietary modification computing system could generate entity-recipe prediction data by calculating multi-dimensional similarities (or dissimilarities) among a first vector representing combined restrictions profile data and taste profile data and a second vector representing uncombined recipe data. As another example, the dietary modification computing system could generate entity-recipe prediction data by calculating multi-dimensional similarities (or dissimilarities) among a third vector representing uncombined restrictions profile data, a fourth vector representing uncombined taste profile data, and a fifth vector representing uncombined recipe data. Additional implementations for generating entity-recipe prediction data based on additional vectors, e.g., representing combined or uncombined data types, could be utilized.

At block 250, the process 200 involves generating optimization data, such as by an entity-recipe relational modification module included in the dietary modification computing system. In some cases, the optimization data identifies one or more modifications to the recipe data, such as modifications to increase the relative effectiveness indicated by the entity-recipe prediction data (or a recalculated value of the entity-recipe prediction data responsive to modifying a portion of the recipe data). In addition, the optimization data identifies a respective impact of a particular data value in the entity taste-restrictions vector on a correlated (e.g., correlated matrix position) data value in the entity recipe-restrictions vector. In some cases, the entity-recipe relational modification module generates the optimization data by applying a high-dimension Jacobian matrix analysis technique to the entity taste-restrictions vector and the entity recipe-restrictions vector, such as by calculating a partial impact of each data value in the entity taste-restrictions vector on the correlated data value in the entity recipe-restrictions vector. In addition, the calculated impact data indicates, for each correlated set of data values, an impact of the data values (or recipe data indicated by the values) on the multi-dimensional similarities (or dissimilarities) among the entity taste-restrictions vector and the entity recipe-restrictions vector. In some cases, the optimization data identifies a particular portion of the recipe data as having relatively high impact data, e.g., modifying particular ingredient data (or other data portion) in the recipe data could increase the relative effectiveness of the recipe data. For example, the entity-recipe relational modification module 150 generates the optimization data 155 by applying a high-dimension vector analysis technique to calculate impact data for each data value in the entity taste-restrictions vector 125 for a correlated data value (e.g., having a correlated matrix position) in the entity recipe-restrictions vector 135.

At block 260, the process 200 involves identifying, by the dietary modification computing system, substitution recipe data associated with one or more portions of the recipe data. In addition, the dietary modification computing system identifies the substitution recipe data based on the respective impact described by the optimization data. For example, in response to determining that the optimization data indicates a relatively high impact, e.g., on the effectiveness of the recipe data, of a particular data value corresponding to a particular portion of recipe data, the entity-recipe relational modification module (or another component of the dietary modification computing system) identifies one or more portions of substitution recipe data that are associated with the particular data value (or the particular portion of recipe data). In some cases, the entity-recipe relational modification module identifies the substitution recipe data based on an ingredient pool data structure. For example, the entity-recipe relational modification module 150 identifies, in the ingredient pool data structure 160, one or more ingredient data sets associated with a particular data value identified by the optimization data 155. In addition, the entity-recipe relational modification module 150 identifies, from the one or more ingredient data sets, a substitution recipe data that can be used by the dietary modification computing system 110 to modify the recipe data 170.

At block 270, the process 200 involves generating, by the dietary modification computing system, modified recipe data. In some cases, the modified recipe data includes a combination of the recipe data and the substitution recipe data. In addition, the entity-recipe relational modification module (or another component of the dietary modification computing system) generates modified recipe data that includes the identified substitution recipe data and omitting recipe data that is replaced by the substitution, e.g., data for an ingredient associated with the impact data indicated by the optimization data. For example, the entity-recipe relational modification module 150 generates the modified recipe data 180 based on a combination of the recipe data 170 with substitution recipe data identified in the optimization data 155.

In some cases, generating the modified recipe data involves receiving, by the dietary modification computing system, additional data received from one or more additional computing systems. For example, the dietary modification computing system provides request data to an additional computing system configured to provide an LLM that can generate data describing a substitution preparation technique for a substitution ingredient. Additionally or alternatively, the dietary modification computing system provides request data to an additional computing system configured to provide an image generation model that can generate substitution presentation data (e.g., image, video) describing a substitution ingredient, substitution preparation technique, or other portions of substitution recipe data. Based on the additional data received from the one or more additional computing systems, the dietary modification computing system generates the modified recipe data to include the data describing the substitution preparation technique, substitution presentation, or other received data.

At block 280, the process 200 involves providing, by the dietary modification computing system, the modified recipe data to one or more additional computing systems. In some cases, the dietary modification computing system provides the modified recipe data to the user device associated with the entity restrictions profile data and the entity taste profile data. In addition, the user device can be configured to present the modified recipe data such as via a user interface device. For example, the dietary modification computing system 110 provides the modified recipe data 180 to the user device 190. In some cases, the dietary modification computing system provides the modified recipe data to an automated ordering computing system, such as an ordering system associated with a grocery store or a restaurant. In addition, the automated ordering computing system can be configured to modify order data to include one or more portions of the modified recipe data, such as to include a substitution ingredient and to omit an ingredient that is replaced by the substitution ingredient.

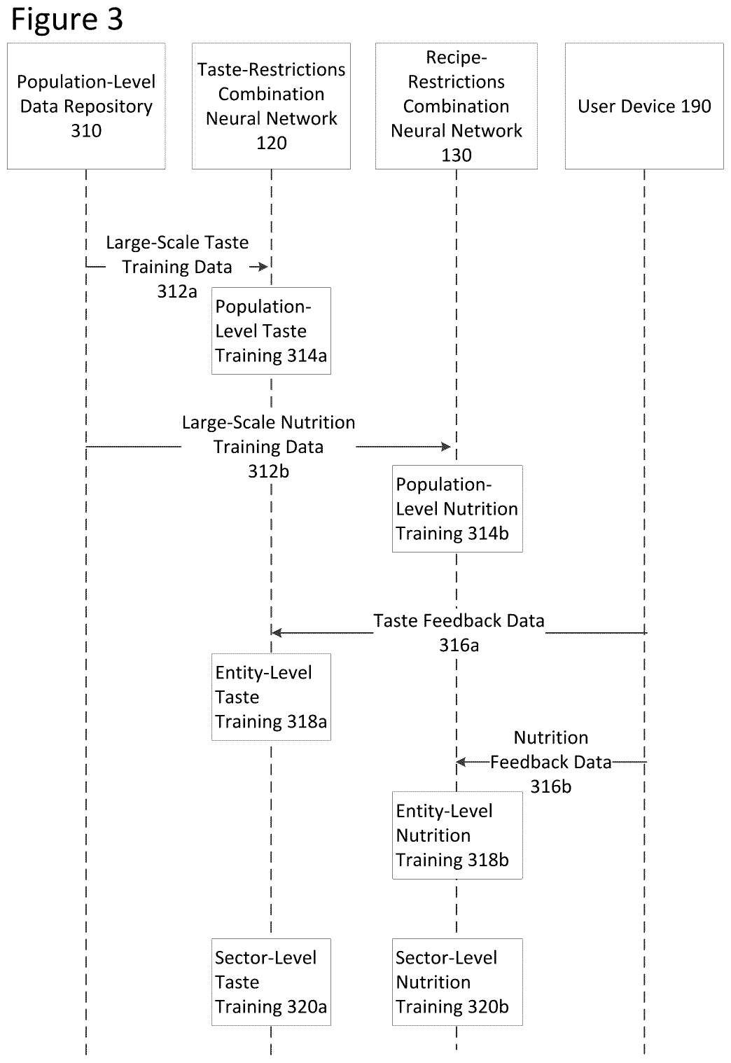

FIG. 3 is a diagram depicting an example of a data flow for one or more training processes for one or more neural networks included in the dietary modification computing system 110. For example, one or more of the taste-restrictions combination neural network 120 or the recipe-restrictions combination neural network 130 can be trained based on at least one of the training processes described in FIG. 3. In some implementations, one or more of the taste-restrictions combination neural network 120 or the recipe-restrictions combination neural network 130 is trained using multi-stage training processes, such as a combination of the population-level training, entity-level training, and sector-level training processes described in FIG. 3. In some cases, multi-stage training processes can improve performance of a neural network in the dietary modification computing system 110, such as increasing accuracy of one or more data outputs generated by the neural network.

In FIG. 3, one or more of the taste-restrictions combination neural network 120 or the recipe-restrictions combination neural network 130 receives training data from a data repository, such as from a population-level data repository 310. In some cases, the population-level data repository 310 is included in, or otherwise provided by, an additional computing system configured to communicate with the dietary modification computing system 110, such as via one or more computing networks as described in regard to the computing environment 100. In addition, the population-level data repository 310 includes one or more data corpus describing one or more of dietary data or taste data across a large population (e.g., tens of thousands, hundreds of thousands) of entities. In FIG. 3, the taste-restrictions combination neural network 120 receives, from the population-level data repository 310, a first portion of large-scale training data at step 312a. In some cases, the first portion of large-scale training data describes large-scale taste preference data, such as data describing generalized enjoyment or dislike (or other preference type) of a particular flavor across the large population of entities. In addition, the recipe-restrictions combination neural network 130 receives, from the population-level data repository 310, a second portion of large-scale training data at step 312b. In some cases, the second portion of large-scale training data describes large-scale nutrition data, such as data describing generalized nutrition requirements across the large population of entities. FIG. 3 describes the taste-restrictions combination neural network 120 and the recipe-restrictions combination neural network 130 as receiving large-scale training data from a particular data repository, such as the population-level data repository 310, but other implementations are possible. For example, the taste-restrictions combination neural network 120 could receive large-scale training data (or additional large-scale training data) from a first data repository that includes large-scale taste training data (e.g., omitting nutrition data). In addition, the recipe-restrictions combination neural network 130 could receive large-scale training data (or additional large-scale training data) from a second data repository that includes large-scale nutrition training data (e.g., omitting taste data).

Based on the first or second portions of large-scale training data received at steps 312a and 312b, the dietary modification computing system 110 trains one or more of the taste-restrictions combination neural network 120 or the recipe-restrictions combination neural network 130. At step 314a, the taste-restrictions combination neural network 120 performs population-level training of at least one machine-learning model (or other artificial intelligence technique) based on the large-scale taste training data. For example, the taste-restrictions combination neural network 120 trains (or modifies training for) the at least one machine-learning model to generate vector representations of the large-scale taste training data combined with additional types of data, such as dietary restriction data. At step 314b, the recipe-restrictions combination neural network 130 performs population-level training of at least one machine-learning model (or other artificial intelligence technique) based on the large-scale nutrition training data. For example, the recipe-restrictions combination neural network 130 trains (or modifies training for) the at least one machine-learning model to generate vector representations of the large-scale nutrition training data (e.g., associated with recipe data) combined with additional types of data, such as dietary restriction data. In addition, one or more of the taste-restrictions combination neural network 120 or the recipe-restrictions combination neural network 130 may verify population-level training, such as by comparing generated vector representations with a validation dataset (e.g., ground truth data). In some cases, population-level training of one or more of the taste-restrictions combination neural network 120 or the recipe-restrictions combination neural network 130 can improve performance of the dietary modification computing system 110, such as by modifying one or more machine-learning model parameters to accurately interpret the large-scale training data.

In FIG. 3, one or more of the taste-restrictions combination neural network 120 or the recipe-restrictions combination neural network 130 receives feedback data from at least one additional computing system, such as the user device 190. In some cases, the generates one or more portions of feedback data in response to receiving data, such as the modified recipe data 180, from the dietary modification computing system 110. At step 316a, the taste-restrictions combination neural network 120 receives one or more portions of taste feedback data from the user device 190. In some cases, the taste feedback data describes entity-level taste preference data, such as whether the person or group of people associated with the user device 190 enjoyed or disliked the meal item described by the modified recipe data 180. At step 316b, the recipe-restrictions combination neural network 130 receives one or more portions of nutrition feedback data from the user device 190. In some cases, the nutrition feedback data describes entity-level nutrition data, such as whether the person or group of people associated with the user device 190 considered the modified recipe data 180 to conflict with a dietary restriction.

Based on the feedback data received at data at steps 316a and 316b, the dietary modification computing system 110 trains one or more of the taste-restrictions combination neural network 120 or the recipe-restrictions combination neural network 130. At step 318a, the taste-restrictions combination neural network 120 performs entity-level training of at least one machine-learning model (or other artificial intelligence technique) based on the taste feedback data. For example, the taste-restrictions combination neural network 120 trains (or modifies training for) the at least one machine-learning model, such as modifying one or more model parameters for generating vector representations of taste preference data combined with dietary restriction data or additional types of data. At step 318b, the recipe-restrictions combination neural network 130 performs entity-level training of at least one machine-learning model (or other artificial intelligence technique) based on the nutrition feedback data. For example, the recipe-restrictions combination neural network 130 trains (or modifies training for) the at least one machine-learning model, such as modifying one or more model parameters for generating vector representations of nutrition data (e.g., associated with recipe data) combined with dietary restriction data or additional types of data.

In some implementations, the dietary modification computing system 110 performs the entity-level training based on calculation of one or more loss functions. For example, such as at the step 318a, the taste-restrictions combination neural network 120 (or another component of the dietary modification computing system 110) generates a taste preference training vector representing the taste feedback data. In addition, the taste-restrictions combination neural network 120 calculates a taste preference loss function that indicates a difference or other relationship between or among the taste preference training vector and the entity taste-restrictions vector 125. Based on the relationship of the taste preference training vector with the entity taste-restrictions vector 125, the taste-restrictions combination neural network 120 modifies the one or more model parameters for generating vector representations of taste preference data combined with dietary restriction data. In some cases, such as at the step 318b, the recipe-restrictions combination neural network 130 (or another component of the dietary modification computing system 110) generates a dietary restrictions training vector representing the nutrition feedback data. In addition, the recipe-restrictions combination neural network 130 calculates a dietary restrictions loss function that indicates a difference or other relationship between or among the dietary restrictions training vector and the entity recipe-restrictions vector 135. Based on the relationship of the dietary restrictions training vector with the entity recipe-restrictions vector 135, the recipe-restrictions combination neural network 130 modifies the one or more model parameters for generating vector representations of recipe data combined with dietary restriction data. In some cases, calculating a loss function based on feedback data can improve performance of one or more of the taste-restrictions combination neural network 120 or the recipe-restrictions combination neural network 130, such as by evaluating accuracy of one or more model parameters in the neural networks 120 or 130.

In some implementations, the dietary modification computing system 110 trains one or more of the taste-restrictions combination neural network 120 or the recipe-restrictions combination neural network 130 based on sector-level data, such as training data or feedback data that is associated with at least one population sector of the large population of entities described by the large-scale training data. In FIG. 3, a population sector (or “sector”) includes a subset of the large population of entities, such as a sector that describes a subset of entities that share a characteristic. Examples of population sectors can include entities that share an age characteristic, a geographical characteristic, a characteristic describing one or more dietary restrictions, or other characteristics (or combinations of characteristics) shared by multiple entities in the large population. In some cases, one or more of the taste-restrictions combination neural network 120 or the recipe-restrictions combination neural network 130 performs sector-level training based on a subset of received data, such as a subset of the large-scale training data received at steps 312a and 312b, a subset of the feedback data received a steps 316a and 361b, or a combination of subsets from the received data. At step 320a, the taste-restrictions combination neural network 120 performs sector-level training of at least one machine-learning model (or other artificial intelligence technique) based on the sector-level subset of received data. For example, based on a sector-level data subset for entities having a particular geographic characteristic, the taste-restrictions combination neural network 120 modifies one or more model parameters related to taste preference data having a geographic characteristic, such as to emphasize or deemphasize vector representations of taste preference data related to a geographic characteristic (e.g., a regional preference for spicy flavors). At step 320b, the recipe-restrictions combination neural network 130 performs sector-level training of at least one machine-learning model (or other artificial intelligence technique) based on the sector-level subset of received data. For example, based on a sector-level data subset for entities having a particular age characteristic, the recipe-restrictions combination neural network 130 modifies one or more model parameters related to data having an age characteristic, such as to emphasize or deemphasize vector representations of nutrition data related to an age characteristic (e.g., a medical recommendation to increase calcium consumption at 55 years of age or older).

In some implementations, the dietary modification computing system 110 performs one or more of sector-level training or population-level training based on calculation of one or more loss functions. For example, the dietary modification computing system 110 (or a component thereof) generates one or more aggregate training vectors representing aggregated feedback data associated with multiple entities in the large population of entities or a sector (e.g., subset) thereof. In addition, the dietary modification computing system 110 (or a component thereof) calculates one or more loss functions indicating a difference or other relationship between or among the aggregate training vectors and one or more of the entity taste-restrictions vector 125 or the entity recipe-restrictions vector 135. Based on the relationship of the one or more aggregate vectors with the entity taste-restrictions vector 125 or the entity recipe-restrictions vector 135, the dietary modification computing system 110 (or a component thereof) modifies the one or more model parameters of the taste-restrictions combination neural network 120 or the recipe-restrictions combination neural network 130.

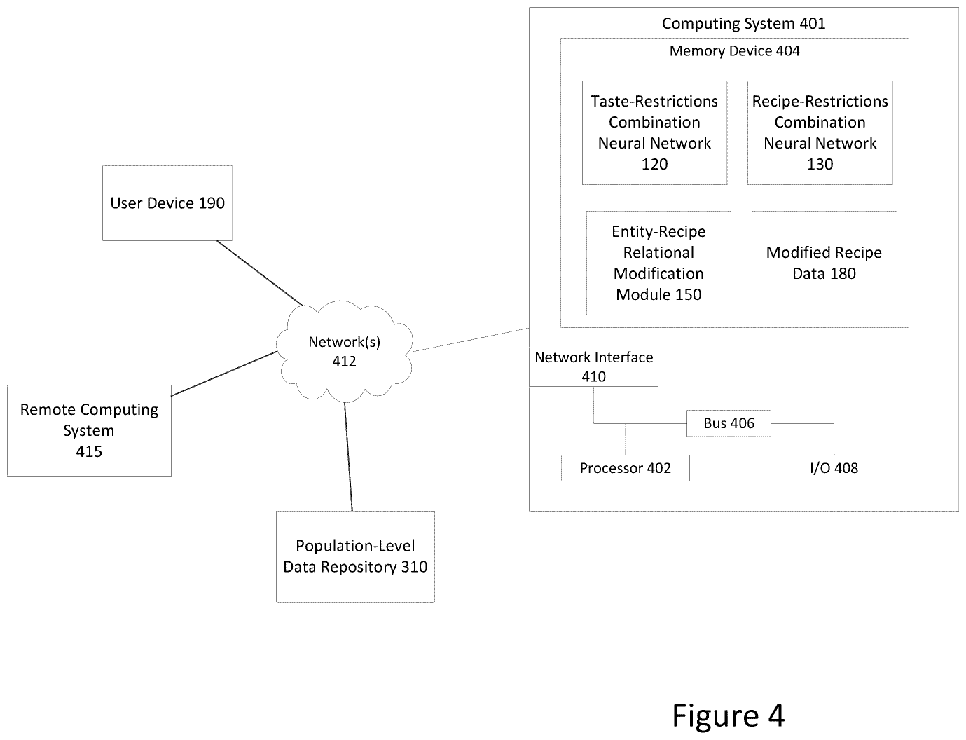

Any suitable computing system or group of computing systems can be used for performing the operations described herein. For example, FIG. 4 is a block diagram depicting an example of a computing system configured to implement a dietary modification computing system, according to certain embodiments.

The depicted example of a computing system 401 includes one or more processors 402 communicatively coupled to one or more memory devices 404. The processor 402 executes computer-executable program code or accesses information stored in the memory device 404. Examples of processor 402 include a microprocessor, an application-specific integrated circuit (“ASIC”), a field-programmable gate array (“FPGA”), or other suitable processing device. The processor 402 can include any number of processing devices, including one.

The memory device 404 includes any suitable non-transitory computer-readable medium for storing the taste-restrictions combination neural network 120, the recipe-restrictions combination neural network 130, the entity-recipe relational modification module 150, the modified recipe data 180, and other received or determined values or data objects. The computer-readable medium can include any electronic, optical, magnetic, or other storage device capable of providing a processor with computer-readable instructions or other program code. Non-limiting examples of a computer-readable medium include a magnetic disk, a memory chip, a ROM, a RAM, an ASIC, optical storage, magnetic tape or other magnetic storage, or any other medium from which a processing device can read instructions. The instructions may include processor-specific instructions generated by a compiler or an interpreter from code written in any suitable computer-programming language, including, for example, C, C++, C#, Visual Basic, Java, Python, Perl, JavaScript, and ActionScript.

The computing system 401 may also include a number of external or internal devices such as input or output devices. For example, the computing system 401 is shown with an input/output (“I/O”) interface 408 that can receive input from input devices or provide output to output devices. A bus 406 can also be included in the computing system 401. The bus 406 can communicatively couple one or more components of the computing system 401.

The computing system 401 executes program code that configures the processor 402 to perform one or more of the operations described above with respect to FIGS. 1-3. The program code includes operations related to, for example, one or more of the taste-restrictions combination neural network 120, the recipe-restrictions combination neural network 130, the entity-recipe relational modification module 150, the modified recipe data 180, or other suitable applications or memory structures that perform one or more operations described herein. The program code may be resident in the memory device 404 or any suitable computer-readable medium and may be executed by the processor 402 or any other suitable processor. In some embodiments, the program code described above, the taste-restrictions combination neural network 120, the recipe-restrictions combination neural network 130, the entity-recipe relational modification module 150, and the modified recipe data 180 are stored in the memory device 404, as depicted in FIG. 4. In additional or alternative embodiments, one or more of the taste-restrictions combination neural network 120, the recipe-restrictions combination neural network 130, the entity-recipe relational modification module 150, the modified recipe data 180, and the program code described above are stored in one or more memory devices accessible via a data network, such as a memory device accessible via a cloud service.

The computing system 401 depicted in FIG. 4 also includes at least one network interface 410. The network interface 410 includes any device or group of devices suitable for establishing a wired or wireless data connection to one or more data networks 412. Non-limiting examples of the network interface 410 include an Ethernet network adapter, a modem, and/or the like. One or more of the user device 190, the population-level data repository 310, or a remote computing system 415 are connected to the computing system 401 via the data networks 412. In some cases, the remote computing system 415 can perform some of the operations described herein, such as storing recipe data or generating entity taste-restrictions vectors or entity recipe-restrictions vectors. The computing system 401 is able to communicate with one or more of the user device 190, the population-level data repository 310, or the remote computing system 415 using the network interface 410. Although FIG. 4 depicts the population-level data repository 310 and the remote computing system 415 as connected to computing system 401 via the data networks 412, other embodiments are possible, including the population-level data repository 310 or the remote computing system 415 (or one or more functions performed by these) running as a program in the memory device 404 of computing system 401.

### General Considerations

Numerous specific details are set forth herein to provide a thorough understanding of the claimed subject matter. However, those skilled in the art will understand that the claimed subject matter may be practiced without these specific details. In other instances, methods, apparatuses, or systems that would be known by one of ordinary skill have not been described in detail so as not to obscure claimed subject matter.

Unless specifically stated otherwise, it is appreciated that throughout this specification discussions utilizing terms such as “processing,” “computing,” “calculating,” “determining,” and “identifying” or the like refer to actions or processes of a computing device, such as one or more computers or a similar electronic computing device or devices, that manipulate or transform data represented as physical electronic or magnetic quantities within memories, registers, or other information storage devices, transmission devices, or display devices of the computing platform.

The system or systems discussed herein are not limited to any particular hardware architecture or configuration. A computing device can include any suitable arrangement of components that provides a result conditioned on one or more inputs. Suitable computing devices include multipurpose microprocessor-based computer systems accessing stored software that programs or configures the computing system from a general purpose computing apparatus to a specialized computing apparatus implementing one or more embodiments of the present subject matter. Any suitable programming, scripting, or other type of language or combinations of languages may be used to implement the teachings contained herein in software to be used in programming or configuring a computing device.

Embodiments of the methods disclosed herein may be performed in the operation of such computing devices. The order of the blocks presented in the examples above can be varied—for example, blocks can be re-ordered, combined, and/or broken into sub-blocks. Certain blocks or processes can be performed in parallel.

The use of “adapted to” or “configured to” herein is meant as open and inclusive language that does not foreclose devices adapted to or configured to perform additional tasks or steps. Additionally, the use of “based on” is meant to be open and inclusive, in that a process, step, calculation, or other action “based on” one or more recited conditions or values may, in practice, be based on additional conditions or values beyond those recited. Headings, lists, and numbering included herein are for case of explanation only and are not meant to be limiting.

While the present subject matter has been described in detail with respect to specific embodiments thereof, it will be appreciated that those skilled in the art, upon attaining an understanding of the foregoing, may readily produce alterations to, variations of, and equivalents to such embodiments. Accordingly, it should be understood that the present disclosure has been presented for purposes of example rather than limitation, and does not preclude inclusion of such modifications, variations, and/or additions to the present subject matter as would be readily apparent to one of ordinary skill in the art.

What is claimed is:

1. A dietary modification computing system comprising:
a taste-restrictions combination neural network configured for:
receiving entity restriction profile data and entity taste profile data; and
generating an entity taste-restrictions vector based on a combination of the entity restriction profile data and the entity taste profile data, the entity taste-restrictions vector including a first high-dimensional data structure in which a first set of data values describe taste-restrictions relationships among the entity restriction profile data and the entity taste profile data;
a recipe-restrictions combination neural network configured for:
receiving the entity restriction profile data and recipe data; and
generating an entity recipe-restrictions vector based on a combination of the entity restriction profile data and the recipe data, the entity recipe-restrictions vector including a second high-dimensional data structure in which a second set of data values describe recipe-restrictions relationships among the entity restriction profile data and the recipe data;
an entity-recipe relational recommendation module configured for:
calculating a degree of multi-dimensional similarity among the entity taste-restrictions vector and the entity recipe-restrictions vector; and
generating entity-recipe prediction data that describes the degree of multi-dimensional similarity;
and an entity-recipe relational modification module configured for:
generating optimization data identifying, for a portion of the recipe data, an impact of the portion of the recipe data on the multi-dimensional similarity among the entity taste-restrictions vector and the entity recipe-restrictions vector;
wherein the dietary modification computing system is configured for:
based on the optimization data, identifying substitution recipe data associated with the portion of the recipe data;
generating modified recipe data that includes a combination of the recipe data with the substitution recipe data, wherein the modified recipe data omits the portion of the recipe data identified by the optimization data; and
providing the modified recipe data to a user computing device.

2. The system of claim 1, wherein:
the entity restriction profile data includes restriction data values describing one or more dietary restrictions associated with an entity, and
the entity taste profile data includes taste preference data values describing one or more taste preferences associated with the entity.

3. The system of claim 1, wherein:
the taste-restrictions combination neural network is prevented from accessing the recipe data, and
the recipe-restrictions combination neural network is prevented from accessing the entity taste profile data.

4. The system of claim 1, wherein the entity-recipe prediction data includes probability data indicating a relative effectiveness of the recipe data in addressing the combination of the entity restriction profile data and the entity taste profile data.

5. The system of claim 1, wherein the entity-recipe relational recommendation module generates the entity-recipe prediction data based on a principal component analysis (“PCA”).

6. The system of claim 1, wherein the entity-recipe relational modification module generates the optimization data responsive to determining that the entity-recipe prediction data fulfills a particular relationship with an optimization threshold value.

7. The system of claim 1, wherein the substitution recipe data is received from an additional computing system configured to provide one or more of a large language model (“LLM”) or an image generation model.

8. A non-transitory computer-readable medium embodying program code, wherein, when executed by a processor, the program code causes the processor to perform operations comprising:
receiving entity restriction profile data, entity taste profile data, and recipe data;
generating an entity taste-restrictions vector based on a combination of the entity restriction profile data and the entity taste profile data, the entity taste-restrictions vector including a first high-dimensional data structure in which a first set of data values describe taste-restrictions relationships among the entity restriction profile data and the entity taste profile data;
generating an entity recipe-restrictions vector based on a combination of the entity restriction profile data and the recipe data, the entity recipe-restrictions vector including a second high-dimensional data structure in which a second set of data values describe recipe-restrictions relationships among the entity restriction profile data and the recipe data;
calculating a degree of multi-dimensional similarity among the entity taste-restrictions vector and the entity recipe-restrictions vector;
generating entity-recipe prediction data that describes the degree of multi-dimensional similarity;
generating optimization data identifying, for a portion of the recipe data, an impact of the portion of the recipe data on the multi-dimensional similarity among the entity taste-restrictions vector and the entity recipe-restrictions vector;
based on the optimization data, identifying substitution recipe data associated with the portion of the recipe data;
generating modified recipe data that includes a combination of the recipe data with the substitution recipe data, wherein the modified recipe data omits the portion of the recipe data identified by the optimization data; and
providing the modified recipe data to a user computing device.

9. The non-transitory computer-readable medium of claim 8, wherein:
the entity restriction profile data includes restriction data values describing one or more dietary restrictions associated with an entity, and
the entity taste profile data includes taste preference data values describing one or more taste preferences associated with the entity.

10. The non-transitory computer-readable medium of claim 8, wherein the entity-recipe prediction data includes probability data indicating a relative effectiveness of the recipe data in addressing the combination of the entity restriction profile data and the entity taste profile data.

11. The non-transitory computer-readable medium of claim 8, wherein the entity-recipe prediction data is generated based on a principal component analysis (“PCA”).

12. The non-transitory computer-readable medium of claim 8, wherein the optimization data is generated responsive to determining that the entity-recipe prediction data fulfills a particular relationship with an optimization threshold value.

13. The non-transitory computer-readable medium of claim 8, wherein the substitution recipe data is received from an additional computing system configured to provide one or more of a large language model (“LLM”) or an image generation model.

14. A method of generating modified recipe data, the method including operations executed by a processor, the operations comprising:
receiving entity restriction profile data, entity taste profile data, and recipe data;
generating an entity taste-restrictions vector based on a combination of the entity restriction profile data and the entity taste profile data, the entity taste-restrictions vector including a first high-dimensional data structure in which a first set of data values describe taste-restrictions relationships among the entity restriction profile data and the entity taste profile data;
generating an entity recipe vector based on the recipe data, the entity recipe vector including a second high-dimensional data structure in which a second set of data values describe the recipe data;
calculating a degree of multi-dimensional similarity among the entity taste-restrictions vector and the entity recipe vector;
generating entity-recipe prediction data that describes the degree of multi-dimensional similarity;
generating optimization data identifying, for a portion of the recipe data, an impact of the portion of the recipe data on the multi-dimensional similarity among the entity taste-restrictions vector and the entity recipe vector;
based on the optimization data, identifying substitution recipe data associated with the portion of the recipe data;
generating modified recipe data that includes a combination of the recipe data with the substitution recipe data, wherein the modified recipe data omits the portion of the recipe data identified by the optimization data; and
providing the modified recipe data to a user computing device.

15. The method of claim 14, wherein:
the entity restriction profile data includes restriction data values describing one or more dietary restrictions associated with an entity, and
the entity taste profile data includes taste preference data values describing one or more taste preferences associated with the entity.

16. The method of claim 14, wherein the entity-recipe prediction data includes probability data indicating a relative effectiveness of the recipe data in addressing the combination of the entity restriction profile data and the entity taste profile data.

17. The method of claim 14, wherein the entity-recipe prediction data is generated based on a principal component analysis (“PCA”).

18. The method of claim 14, wherein the optimization data is generated responsive to determining that the entity-recipe prediction data fulfills a particular relationship with an optimization threshold value.

19. The method of claim 14, wherein the substitution recipe data is received from an additional computing system configured to provide one or more of a large language model (“LLM”) or an image generation model.
